(19) United States
(12) Patent Application Publication
(10) Pub. No.: US 2006/0025449 A1
(43) Pub. Date: Feb. 2, 2006

(54) Use of N-substituted imino sugars for appetite suppression

(75) Inventors: David A. Priestman, Oxford (GB); Aarnoud C. van der Spoel, Oxfordshire (GB); Terry D. Butters, Oxford (GB); Raymond A. Dwek, Oxford (GB); Frances M. Platt, Long Hanborough (GB)

Correspondence Address: FOLEY AND LARDNER LLP;SUITE 500, 3000 K STREET NW, WASHINGTON, DC, 20007, US

(73) Assignee: UNITED THERAPEUTICS

(21) Appl. No.: 11/086,654

(22) Filed: Mar. 23, 2005

Related U.S. Application Data
(60) Provisional application No. 60/556,054, filed on Mar. 25, 2004.

(51) Int. Cl. A61K 31/445

(52) U.S. Cl. 514/328

(57) ABSTRACT

The present invention relates to compositions and methods for treating obesity. In particular, this invention describes the use of imino sugars, such as N-butyldeoxynojirimycin (NB-DNJ) for appetite suppression, thereby causing weight loss.

### PRIORITY

This application claims priority to U.S. Provisional Application No. 60/556,054, filed on Mar. 25, 2004, which is incorporated herein by reference in its entirety.

### BACKGROUND

1. Field of the Invention

This invention relates to the use of imino sugars to treat obesity. In particular, this invention describes the use of N-butyldeoxynojirimycin (NB-DNJ) for appetite suppression, thereby causing weight loss.

2. Background of the Invention

Obesity is a public health problem which is both serious and widespread: in industrialized countries, a third of the population has an excess weight of at least 20% relative to the ideal weight. The phenomenon continues to worsen in regions of the globe whose economies are being modernized, such as the Pacific islands, and in general. In the United States, the number of obese people has passed from 25% at the end of the 70 s to 33% at the beginning of the 90 s.

Obesity is associated with increased morbidity and mortality. It has been linked to a number of diseases including type 2 diabetes mellitus, hypertension, coronary artery disease, stroke, hypercholesterolemia, cholelithiasis, fatty liver disease, certain cancers (postmenopausal breast cancer and cancers of the colon, endometrium and kidney), musculoskeletal disorders (osteoarthritis), obstructive sleep apnea, and infertility, not to mention the social consequences and isolation that many patients with obesity experience. Department of Agriculture, Department of Health and Human Services. Nutrition and Your Health: Dietary Guidelines for Americans, 4^{th} Ed. Home and Garden Bulletin No. 232. Washington, D.C.: Government Printing Office, 1995.

Whether the physiological changes in obesity are characterized by an increase in the number of adipose cells or by an increase in the quantity of triglycerides stored in each adipose cell, or by both, this excess weight results mainly from an imbalance between the quantities of calories consumed and those of the calories used by the body. Studies on the causes of this imbalance have been in several directions. Some have focused on studying feeding behavior, and therefore the molecules and hormones which control food intake and feelings of hunger/satiety. Other studies have been related to basal metabolism, that is to say the manner in which the body uses the calories consumed.

Current treatment strategies have been disappointing and largely ineffective for long-term success. Certainly every effort is made to set short-term goals and recognize the importance of lifestyle alterations in the form of increased exercise and decreased caloric intake. Drug therapy is now limited since fenfluramine and dexfenfluramine have been taken off the market due to their possible link to valvular heart disease. Two new agents, sibutramine (Meridia), a catecholaminergic and serotonergic agonist, and orlistat (Xenical), a lipase inhibitor, have recently been approved by the FDA. Unfortunately, sibutramine causes dry mouth, headaches, insomnia, constipation, and dose related increases in heart rate and blood pressure (see Sibutramine for Obesity, The Medical Letter, 40:32 (1998)). Orlistat can cause flatulence, oily stools, and fecal urgency and interferes with the absorption of the fat soluble vitamins (A, D, E, and beta carotene). (see Orlistat for Obesity, The Medical Letter, 41:55-6 (1999)). Both drugs are only indicated for short term treatment and weight gain after cessation is common. Additionally, Sibutramine and Orlistat both now have FDA approval for long-term use and a wide variety of new targets for obesity treatment are in development (see Komer and Aronne, J Clin Invest., 111 (5), 565-70 (2003)).

Additionally, surgical therapy is reserved for patients with severe obesity, or those patients with lesser obesity that have coexisting conditions. Jejunal-ileal shunting can be effective but it is costly and frequently results in symptoms related to a blind loop. The more common gastroplasty procedure is also costly and can cause “dumping” associated with the passage of gastric contents into the intestine. As such, these patients need to be followed carefully for intestinal obstruction and electrolyte disturbances. Excess consumption of liquid or semisolid foods can negate the benefits of both procedures. Browenll K D, Fairbum C G, eds. Eating Disorders and Obesity: A Comprehensive Handbook. New York: Guilford Press, 1995.

Therefore, there is a need in the art for alternative methods and compositions for treating obesity. The present invention addresses that need. Indeed, the inventors determined that an N-substituted imino sugar, N-butyldeoxynojiri-mycin (NB-DNJ) in particular, induces appetite suppression, decreases caloric intake, and therefore causes weight loss in obese mice.

### SUMMARY OF THE INVENTION

The present invention describes compositions for treating obesity comprising an N-substituted imino sugar and a pharmaceutically acceptable excipient. In one embodiment, the N-substituted imino sugar is N-butyldeoxynojirimycin.

Also described herein are methods for treating obesity comprising administering an effective amount of an N-substituted imino sugar that does not cause an initial hyperphagic response, and a method for centrally suppressing an appetite comprising administering an effective amount of an N-substituted imino sugar to a subject, for example, one that is obese. In one embodiment, the N-substituted imino sugar is N-butyldeoxynojirimycin. In another embodiment, the N-butyldeoxynojirimycin causes a reduction in food intake sufficient to cause weight loss.

The present application also describes a method for depleting white adipose tissue in a subject comprising administering an effective amount of N-butyldeoxynojirimycin. In one embodiment, the subject is obese.

### BRIEF DESCRIPTION OF THE DRAWINGS

FIG. 1. Effect of Imino sugars on the growth of C57B1/6 Mice fed 2400 mg/kg/day NB-DNJ, NB-DGJ, or a control diet. (n=10 per group).

FIG. 2. Effect of NB-DNJ on mouse skin adipose tissue. 6 week-old Mice NB-DNJ-treated for 4 weeks with 2400 mg/kg/d. Mice were sacrificed and hair removed by shaving followed by application of a depilatory cream. A small section of skin was excised from the same area of the back of each mouse and processed for histology. (See example 5 for further details)

FIG. 3. Dose-response effect of NB-DNJ on epididymal fat pads. Mice (n=5 per group) were treated with NB-DNJ for 5 weeks from 6 weeks old. Epididymal fat pads were then excised and weighed in pairs. Weights are expressed as a ratio to total body weight.

FIG. 4. Growth curves of control and obese mice. Mice were maintained on diet with or without NB-DNJ, 2400 mg/kg/d, for up to six months of age. C57B1/6 mouse group numbers: n=12 to day 75, n=6 to day 112, n=3 to day 190. To study the effect of compound on obese mice, three groups (n=5) of ob/ob mice were monitored from five weeks of age. Two groups started on control diet and were maintained on this diet up to 83 days of age. One of the groups was supplied with NB-DNJ thereafter.

FIG. 5. Growth curves for Control and NB-DNJ-treated mice. Growth of the control mice with and without NB-DNJ treatment excluding the portion of the curve where the treated mice lost weight (see FIG. 4). The data were analysed by univariate analysis of variance (ANOVA) to compare the slopes of growth statistically. SPSS software was used to process the data.

FIG. 6. Tibia lengths in Control and NB-DNJ-treated mice. The effect of NB-DNJ on lean body mass growth was assessed by measuring tibia lengths in a group of 20 control mice at one year old, 5 mice at 2 years old and two mice 19.5 months old after 18 months on 2400 mg/kg/day. Tibias were dissected from both hind-limbs then boiled in distilled water for 20 min to clean the bone of remaining connective tissue and muscle. Tibia length was then measured using calipers.

FIG. 7. Dietary Intake in lean control and obese mice. A known amount of diet was provided to groups of 5 mice per cage. Mice were allowed to feed ad libitum and diet was weighed and replenished on a daily basis for 7 days. For treatment with imino sugar in the, mice were given 2400 mg/kg/day equivalent NB-DNJ admixed with diet or in isotonic saline for intraperitoneal injections.

FIG. 8. Growth curves for mice injected intraperitoneally with saline vehicle/NB-DNJ, on restricted diet and with minipumps. Groups of lean control and obese mice (n=5) were allowed to feed on a pelleted diet ad libitum. One group each (lean/obese) were given intraperitoneal injections containing 48 mg NB-DNJ in saline. Controls received saline vehicle. Two weeks into the study, two obese mice had minipumps implanted which delivered 2.6 mg NB-DNJ per day for 28 days. Thereafter, these two mice received intraperitoneal injections as previously described. Dietary intake was assessed for the mice receiving injections. Then, one group each of control and obese mice received a restricted diet equivalent to that eaten by the ip injected groups.

FIG. 9. Dietary intake in 24 hours following intracerebroventricular injection of 2 nmol NB-DNJ, NB-DGJ, 2-deoxyglucose or 1 μl sterile saline. Student's t-test was used to calculate p-values

FIG. 10. Dietary intake and weight change in 24 hours following intracerebroventricular injection of 2 nmol NB-DNJ, NB-DGJ, 2-deoxyglucose or 1 μl sterile saline. Student's t-test was used to calculate p-values

FIG. 11. Control lean mice (n=5 in each group) had icv minipumps (50 mM imino sugar) implanted. Body weight was the monitored at regular intervals over three weeks. See example 7 for further experimental details.

FIG. 12. Obese mouse growth with ICV minipump implants. Two groups (n=5) of obese mice were implanted with icv minipumps containing sterile isotonic saline or 100 mM NB-DGJ. They were then weighed at regular intervals for four weeks

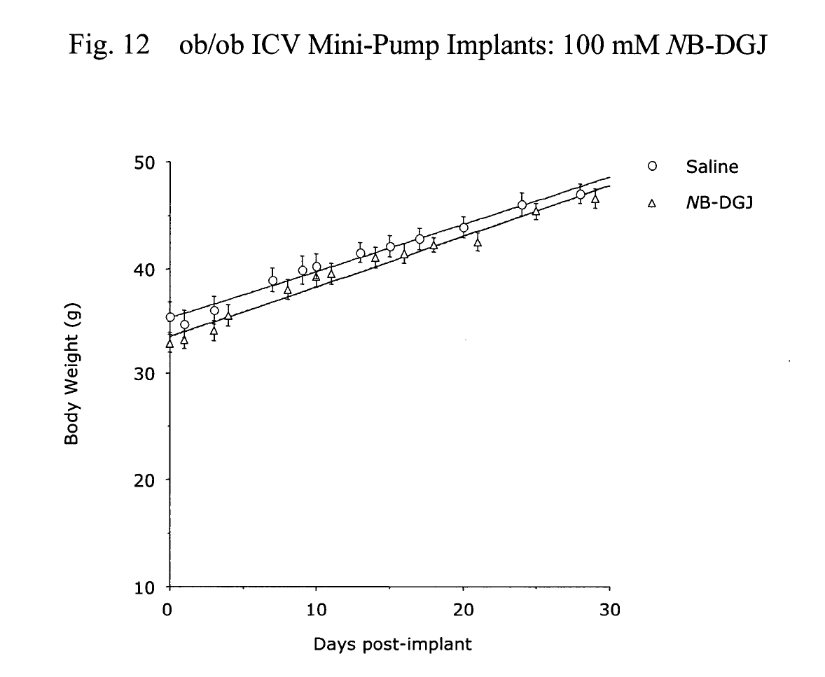

### DETAILED DESCRIPTION OF THE INVENTION

Overview

The molecular regulation of body weight and feeding behavior is a highly complex process and recent discoveries regarding neuronal circuits and their hormonal regulation are leading to a much greater understanding of energy homeostasis. The discovery of leptin and a number of other gene products in rodent models has shown that similar mechanisms are involved in body weight regulation across different mammalian species. To date, there are at least sixteen different hormones, neurotransmitters and peptides implicated in the inhibition of feeding behavior in rodents (Ahima & Osei, Trends Mol Med 7, 205-13 (2001)). Intracerebroventricular injections have confirmed a central appetite suppression effect for all these factors.

The imino sugar NB-DNJ also has a variety of inhibitory activities. These include inhibition of the N-linked glycan processing enzymes α-glucosidases I and II (Fischer et al., J Virol 69, 5791-7 (1995); Fischer et al., J Virol 70, 7153-60 (1996)), the ceramide-specific glucosyl-transferase involved in glycosphingolipid (GSL) biosynthesis (Platt et al., J Biol Chem 269, 8362-5 (1994)), the disaccharidases, sucrase and maltase (Andersson et al., infra), and glycogen debranching enzyme (Andersson et al., Biochemical Pharmacology 67, 697-705 (2004)). As an inhibitor of GSL biosynthesis, NB-DNJ is presently in clinical trials as a potential treatment for GSL storage diseases with a neurological component, and is approved for use in type 1 Gaucher disease in Europe, Israel and the USA (Cox et al., Lancet 355, 1481-5 (2000); Lachmann et al., Curr Opin Investig Drugs 4, 472-9 (2003)).

In the present invention, the inventors discovered that NB-DNJ has a central anorectic effect, causing central appetite suppression, depletion of white adipose tissue, and therefore weight loss. While adverse side effects associated with NB-DNJ administration to both mice and humans include significant weight loss seen in mice on relatively high doses of the drug and in some individuals in the human clinical trials (Platt et al., J Biol Chem 272, 19365-72 (1997)and Platt and Lachmann, 2001), the ability of this N-substituted imino sugar has not been previously reported to induce appetite suppression in obese subjects or its ability to treat obesity. Indeed, Platt does not show that NB-DNJ specifically causes weight loss, or if the weight loss was a non-specific effect caused by the drug reaching its toxic upper limit. Additionally, the inventors were the first to identify a specific effect of NB-DNJ, i.e., its ability to act centrally to cause weight loss.

Definitions

As described herein, an “effective amount” means an amount sufficient to cause a particular effect, such as reduced growth, weight loss or depletion of white adipose tissue.

The term “obesity” connotes an increase in body weight beyond the limitation of skeletal and physical requirement, as the result of an excessive accumulation of fat in the body. As described herein, an obese subject has a body mass index of greater than 30, an overweight subject, greater than 25. Body mass index is usually calculated by weight (lbs)×704/(height (in))² or weight (kg)/(height (m))².

Compositions and Methods of Treatment

The instant invention describes compositions for treating obesity comprising an N-substituted imino sugar and a pharmaceutically acceptable excipient. In a preferred embodiment, the imino sugar is NB-DNJ.

Also contemplated in the present invention are methods for treating obesity comprising administering an effective amount of an N-substituted imino sugar that does not cause an initial hyperphagic response. In one embodiment, the imino sugar is NB-DNJ. In another embodiment, the imino sugar is administered in 100 mg dosages once, twice or three times daily.

Also disclosed in the present invention are methods for depleting adipose tissue mass comprising administering an effective amount of an N-substituted imino sugar. In one embodiment, the imino sugar is NB-DNJ.

Similarly, described herein is a method for centrally suppressing an appetite comprising administering an effective amount of an N-substituted imino sugar. Preferably, the sugar is NB-DNJ. Also preferred, NB-DNJ caused at least a 10% reduction in food intake. More preferably, a 20% reduction, and most preferably, a 30% reduction in food intake.

Administration of the composition described herein during treatment may be by any number of routes, including parenteral and oral, but preferably parenteral. For example, intracapsular, intravenous, intrathecal, and intraperitoneal routes of administration may be employed. The skilled artisan will recognize that the route of administration will vary depending on the disorder to be treated.

Determining a therapeutically effective amount of the compositions of the present invention largely depend on particular patient characteristics, route of administration, and the nature of the disorder being treated. General guidance can be found, for example, in the publications of the International Conference on Harmonisation and in REMINGTON'S PHARMACEUTICAL SCIENCES, chapters 27 and 28, pp. 484-528 (Mack Publishing Company 1990).

Determining a therapeutically effective amount specifically will depend on such factors as toxicity and efficacy of the medicament. Toxicity may be determined using methods well known in the art and found in the foregoing references. Efficacy may be determined utilizing the same guidance in conjunction with the methods described below in the Examples. A pharmaceutically effective amount, therefore, is an amount that is deemed by the clinician to be toxicologically tolerable, yet efficacious. Efficacy, for example, can be measured by the decrease in mass of the targeted tissue. As described above, suitable dosages can be from about 100 mg/day.

The mechanism of NB-DNJ-mediated weight loss is primarily due to appetite suppression and mice experience similar weight loss or lack of weight gain when fed a restricted diet that mimics the drug-induced level of food consumption. The weight loss does not appear to result from inhibition of either disaccharidases in the gut or enzymes of glycogenolysis. Instead, mice injected intracerebroventricularly with NB-DNJ exhibited weight loss as a result of reduced food consumption confirming a centrally acting mechanism of weight loss.

Thus, the present invention also contemplates methods for screening other imino sugars that may be suitable for treating obesity. Such compounds will act via a mechanism similar to NB-DNJ and have activity similar to NB-DNJ. NB-DNJ activity can be easily assessed by employing any of the methods described herein. For example, intracerebroventricular injections followed by assessment of dietary intake and weight change provide an assay for confirming a central effect in causing appetite suppression. Also, this system only requires very small quantities of test substances.

In addition, without wishing to be bound to any theory, the inventors believe that NB-DNJ may induce feeding suppression via a mechanism similar to 1,5-anhydroglucitol because the hydroxyl group on the glucopyranose ring of 1,5-anhydroglucitol, particularly at carbon 1 or 2, is involved in feeding modulation (Sakata & Kurokawa, Am J Clin Nutr 55, 272S-277S (1992)). Indeed, NB-DNJ, is a glucose analogue lacking a hydroxyl group on carbon 1. But in contrast to the action of 2-deoxyglucose by intraventricular infusion, which induced feeding suppression over three days (Tsutsui et al., Physiol Behav 31, 493-502(1983)), NB-DNJ did not cause an initialhyperphagic response in the first 24 hrs post icv injection.

Pharmaceutically Suitable Excipient

The imino sugars of the present invention can be formulated according to known methods to prepare pharmaceutically useful compositions, whereby the inventive molecules, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (16th ed., Osol, A., ed., Mack, Easton Pa. (1980)). To form a pharmaceutically acceptable composition suitable for effective administration, such compositions will contain an effective amount of one or more of the proteins of the present invention, together with a suitable amount of carrier vehicle.

The compositions for use in accordance with the present invention may be formulated in conventional manner using one or more physiologically acceptable carriers or excipients. Thus, the compositions described herein may be formulated for administration by inhalation or insufflation (either through the mouth or the nose) or oral, buccal, rectal or other formulations for parenteral administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they maybe presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the composition may take the form of tablets or lozenges formulated in conventional manner.

For administration by inhalation, the imino sugars according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g. gelatin for use in an inhaler or insufflator may be formulated containing a powder mix of the compound and a suitable powder base such as lactose or starch.

The imino sugars of the present invention may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the imino sugars of the present invention may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The invention is further described by reference to the following examples, which are provided for illustration only. The invention is not limited to the examples but rather includes all variations that are evident from the teachings provided herein.

### EXAMPLES

### Example 1

### Materials and Methods

NB-DNJ was a gift from the Monsanto/Searle Company and Oxford GlycoSciences (Abingdon, Oxfordshire, UK) and NB-DGJ was purchased from Toronto Research Biochemicals (Downsview, ON, Canada). 2-deoxyglucose was purchased from Sigma (Poole, UK).

### Example 2

### Treatment of Mice with Imino Sugars

Control C57B1/6 and C57BL/6O1aHsd-Lep ob mice were obtained from Harlan, UK at five weeks of age. The mice were housed under standard non-sterile conditions and fed a diet of mouse chow pelleted or expanded ground RM1 diet (SDS Ltd, Witham, Essex) with water available ad libitum. For dietary treatment of the mice with NB-DNJ or NB-DGJ, the diet and compound were admixed thoroughly as dry solids, stored at room temperature and used within 7 days. Unless indicated otherwise, mice were treated with a dose of 2400 mg/kg body weight per day, assuming an intake of around 5 g diet/day. Mice were maintained on diet with or without compound for up to six months of age and were weighed at regular intervals during the course of the experiment (see figure legends for group sizes and further experimental details). To study the effect of compound on obese mice, three groups (n=5) of ob/ob mice were monitored from five weeks of age. One group acted as control and were fed ground RM1 diet. At 6 weeks of age, the second group was started on NB-DNJ in the diet, whilst the third group started drug treatment at 12 weeks of age. All mice were then monitored for up to six months of age.

Growth rates of control and NB-DNJ-treated mice were analysed statistically by univariate analysis of variance (ANOVA) using SPSS software to compare slopes of growth from 54 to 174 days of age.

The effect of NB-DNJ on lean body mass growth was also assessed by measuring tibia lengths in a group of 20 control mice at one year old, 5 mice at 2 years old and two mice 19.5 months old after 18 months on 2400 mg/kg/day. Tibias were dissected from both hind-limbs then boiled in distilled water for 20 min to clean the bone of remaining connective tissue and muscle. Tibia length was then measured using calipers.

Dietary intake of mice on powdered diets was measured on a daily basis, by pre-weighing diet put into the feeding hoppers then weighing remaining diet daily. Diet which the mice had spilt on the floor of the cage, was carefully recovered by sieving and incorporated into the measurements.

### Example 3

### Intraperitoneal Injections and Dietary Restriction

In order to facilitate measurement of dietary intake, to exclude possible effects of the bitter taste of imino sugar in the diet and also inhibitory effects on disaccharidases in the gastrointestinal tract, groups of control and obese mice five weeks old were given daily intraperitoneal injections of NB-DNJ at a dose equivalent to 2400 mg/kg/day. A further group of obese mice was given daily intraperitoneal injections for two weeks from 10 weeks old. Control mice for each group were injected with an equal volume of isotonic saline vehicle. After assessment of dietary intake over five weeks in controls and three weeks in the obese mice, a restricted diet of 3.2 g/day for controls and 4.0 g/day for the obese mice was provided to compare with the effect of intraperitoneal injection. This equated to a 30% reduction in dietary provision for both groups of mice.

That the intraperitoneally injected mice eat 30% less than their age and sex-matched controls, suggests that both the bitter taste of imino sugars and possible effects on gut enzymes are unlikely to be factors in NB-DNJ-induced weight loss. It also supported the suggestion that there may be a central mechanism causing appetite suppression. The effect of NB-DNJ on lean mouse growth concurred with previous studies. Andersson et al., Biochem Pharmacol 59, 821-9 (2000); Platt et al. J Biol Chem 272, 19365-72 (1997).

### Example 4

### Mini-Osmotic Pumps

Alzet mini-osmotic pumps (model 2004, DURECT Corporation, CA, USA) containing 2M NB-DNJ were implanted subcutaneously in the flanks of control and obese mice under isofluorane anaesthesia (4% for induction, 2.5%, maintenance).

### Example 5

### Skin Histology/Thickness

Six week-old control mice on a normal diet or treated with 2400 mg/kg/day NB-DNJ for four weeks (n=3 in each group), were sacrificed and hair removed by shaving followed by application of a depilatory cream. A small section of skin was excised from the same area of the back of each mouse and processed for histology. After excision, the skin was put into mini histology trays containing OCT compound (BDH Laboratory Supplies, UK) and frozen for cryo-sectioning. Cryosections (10 μm thickness) of skin were then fixed in ice-cold acetone for 30 sec and rinsed under running tap water for 1 minute before dipping briefly in Mayer's haemotoxylin (Sigma, UK). The sections were dehydrated through a rising ethanol concentration series of 70%, 80%, 95%, 100% before clearing with Histo-Clear Histological Clearing agent (National Diagnostics, Hessle, Hull, UK) twice for 5 min. Finally, the sections of skin were mounted with cover slips and DePeX mounting medium (BDH Laboratory Supplies, UK) on glass microscope slides.

Another portion of skin (5 mm²) was taken from each mouse, placed on a microscope slide and skin thickness measured using a micrometer gauge.

### Example 6

### Epididymal Fat Pads

A dose-response relationship for the effect of increasing concentrations of NB-DNJ on adipose tissue in male C57B1/6 mice was determined by treating with doses ranging from 15 to 2400 mg/kg/day for 5 weeks. Mice were sacrificed and their epididymal fat pads excised and weighed. The results are expressed as a ratio of epididymal pad fat mass to total body weight.

### Example 7

### Central Anorexigenic Mechanism

Intracerebroventricular Injections

To investigate a central anorexigenic mechanism for weight loss in mice treated with NB-DNJ, single intracerebro-ventricular (ICV) injections were performed. The mice were assessed for food intake and weight change.

Mice were anesthetized with isofluorane (4%) delivered by a vaporiser. Upon achievement of a surgical plane of anaesthesia, the animal was transferred to and secured in a Kopf small animal ultra precise stereotaxic instrument (David Kopf Instruments, Tujunga, Calif., USA) fitted with rat ear bars, mouse adaptor and mouse anaesthesia mask. A small amount of lignocaine local anaesthetic gel was applied to the ear bars to reduce any post-operative discomfort. For the remainder of the procedure, anaesthesia was maintained with 2.5% isofluorane. Lacrilube (Allergan Ltd, High Wycombe, UK) was applied to the mouse's eyes and a subcutaneous injection of the non-steroidal anti-inflammatory drug, Rimadyl (Pfizer, Sandwich, Kent, UK) was given for post-operative analgesia. For ICV injections, a small scalpel incision was made along the midline of the scalp from above the eyes to the occiput. A small hole was drilled in the skull, 1 mm lateral and 0.34 mm posterior to Bregma. 1 μl solution was slowly infused into the lateral ventricle at a depth of 2.25 mm, via a glass needle pipette pulled to a thickness of less than 100 microns at the tip. Mice were injected with 2 nmol compound (NB-DNJ, NB-DGJ or 2-deoxy glucose) in isotonic saline vehicle which was injected into controls. The incision was then closed using VetBond acrylic suture fluid (3M Animal Care Products, St Paul, Minn., USA). The whole procedure from induction of anaesthesia to full recovery lasted less than seven minutes. Mice were allowed free access to a pre-weighed quantity of RM1 pellet diet with water provided ad libitum. 24 hours following ICV injection the mice and remaining diet were weighed.

ICV Mini-Pump Implants

The effect of long-term intracerebro-ventricular delivery of imino sugars was determined by the implantation of Alzet mini-osmotic pumps (model 2004, DURECT Corporation, CA, USA) together with the brain infusion kit II which features a high stability low profile cannula. The mini-pumps were loaded with 0.24 ml imino sugar or saline vehicle. The indwelling 28 gauge stainless steel cannula was cut to a length of 2.25 mm. Mice were anaesthetised as for the ICV injections and underwent similar surgical procedures throughout. After drilling a small hole in the cranium at the midline, 2 mm posterior to Bregma, the mini-pump was positioned subcutaneously in the animal's flank and the cannula was implanted. This position and depth ensured delivery to the third cerebral ventricle. The implant was secured to the cranium using VetBond acrylic suture fluid, which was also used to close the surgical wound. The whole procedure lasted less than 9 minutes and mice were fully conscious and recovered within three minutes. For concentration of imino sugar in mini-pumps see figure legends.

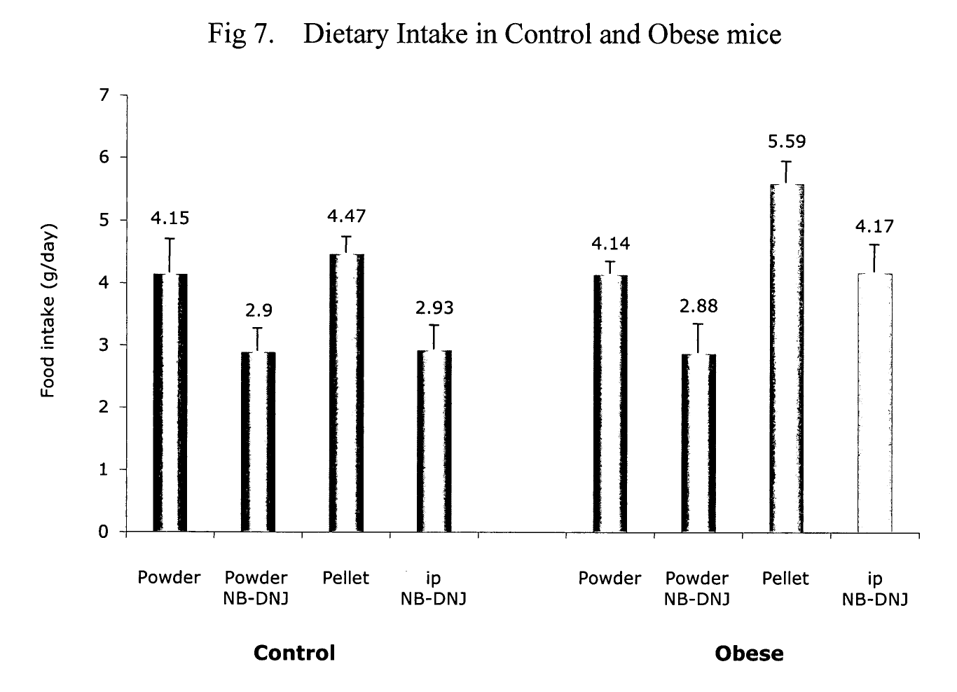

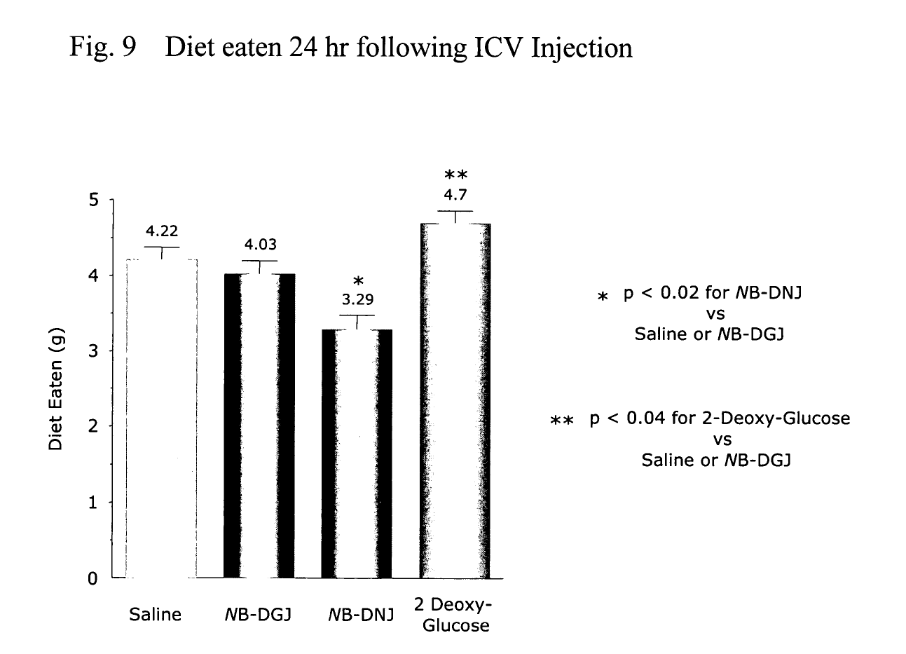

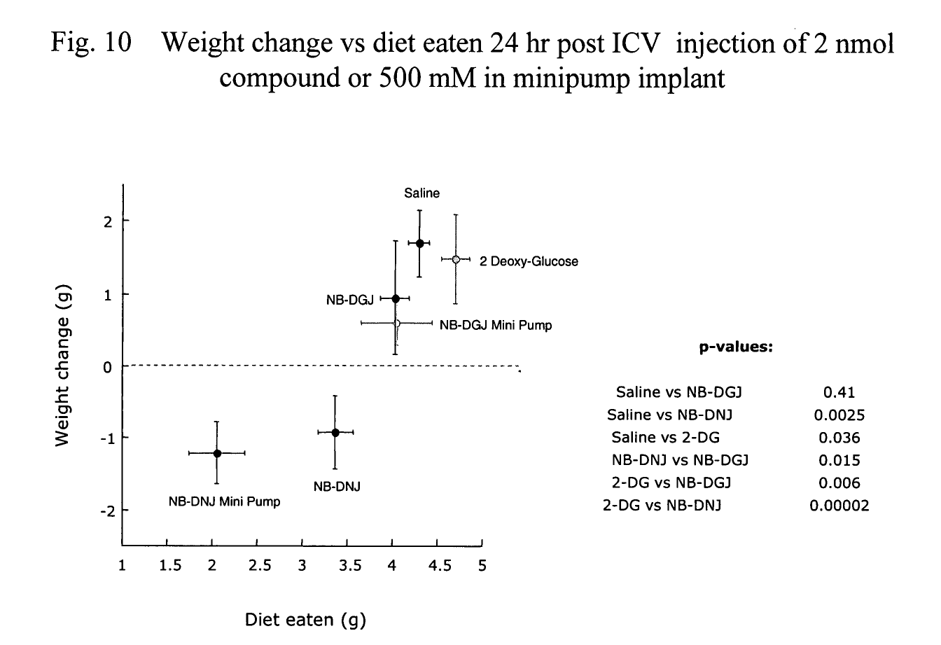

To confirm a central anorectic effect of NB-DNJ, 2 nmol NB-DNJ, NB-DGJ and 2 deoxyglucose were injected directly into the lateral cerebral ventricle of lean control mouse brain (approximately 100 μM final cerebro-ventricular concentration). Mice injected with both saline vehicle and NB-DGJ ate a normal quantity of pellet diet in the following 24 hours (FIG. 9), approximately 5% less in this experiment than that seen in control mice in FIG. 7 (not statistically significant). In contrast, NB-DNJ-injected mice ate 20% less (p<0.02 for both saline and NB-DGJ) and 2-deoxy-glucose injections caused a 10% increase in feeding. 2-deoxy-glucose is a mild appetite stimulant in the short-term. Tsutsui et al., Physiol Behav 31, 493-502. In contrast, NB-DGJ had no effect, as expected. This was also confirmed with the ICV mini-pump implants which were loaded with 500 mM NB-DGJ, which had no statistically significant effect on either dietary intake or weight change (FIG. 10). This figure also shows the significant weight loss, caused by the reduction in food intake in the 24 hours following ICV injection of NB-DNJ.

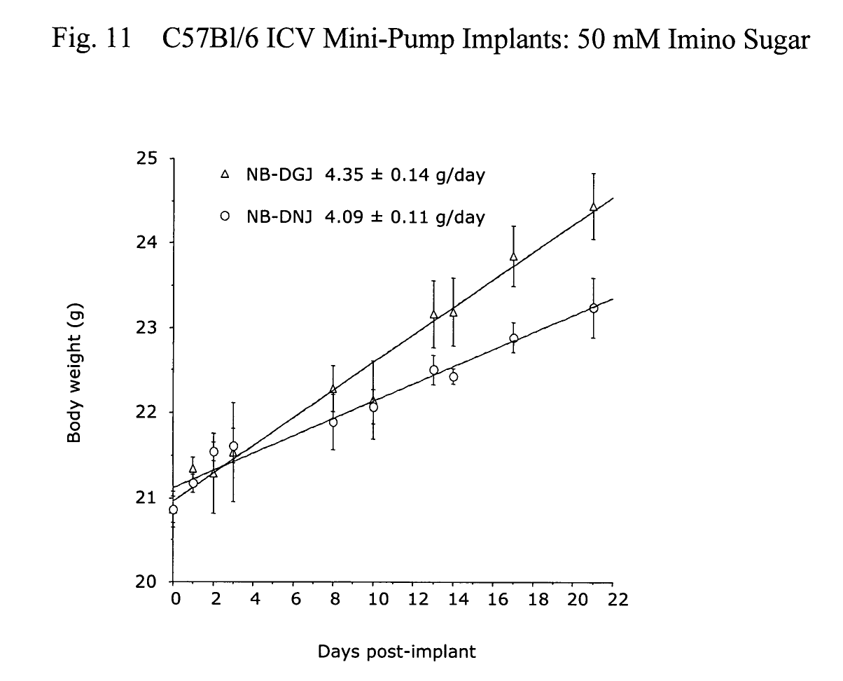

Mice with ICV mini-pump implants containing 500 mM AB-DNJ ate even less and lost a little more weight in the first 24 hours, and in a subsequent experiment, the concentration of NB-DNJ in the ICV mini-pump was reduced to 50 mM and NB-DGJ was again used as a control (FIG. 11). A 6% reduction in food intake, compared to the controls, caused a 5% reduction of body weight after 3 weeks.

### Example 9

### Effect of Imino Sugars on Mouse Growth

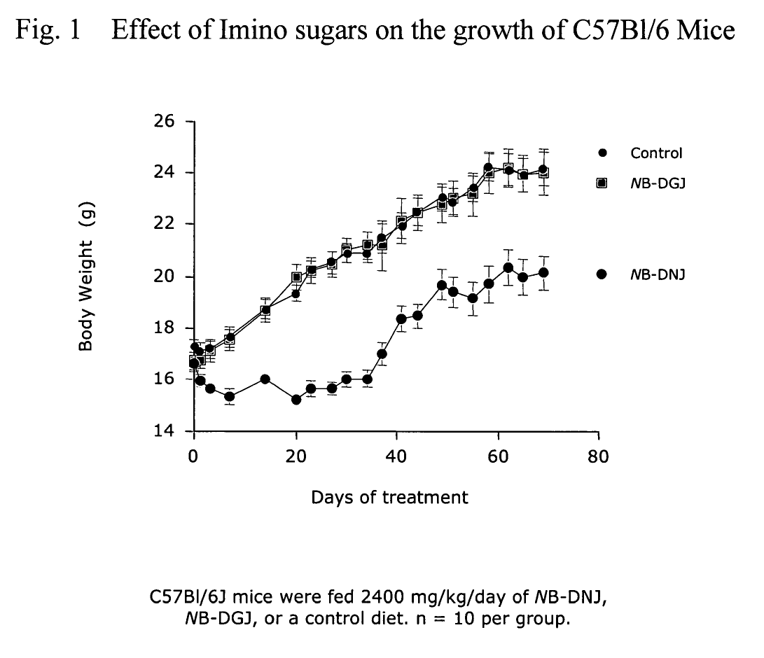

FIG. 1. shows the effect of both NB-DNJ and NB-DGJ on the growth of normal control mice from 6 weeks of age. In the first week of treatment, mice with NB-DNJ in their diet lost about 10% of their total body mass. Over the course of the following weeks, their weight stabilises. By 5 weeks of treatment they weighed 25% less than the controls. They then started to gain weight. However, after 10 weeks they remained 20% less in mass when compared with the age and sex-matched control group. In contrast to the effect of NB-DNJ on growth, NB-DGJ has no discernible effect and mice follow the same pattern of growth as the untreated controls.

The lack of any effect of NB-DGJ also implies that the significant inhibition of GSL biosynthesis caused by both imino sugars played no role in mouse growth. NB-DGJ could therefore be effectively used as a control compound when studying the effects of NB-DNJ on body weight of mice. Whilst a well-defined effect on total body weight has only been seen in doses of 600 mg/kg/day and above (Platt et al.), a reduction in fat pad weight is apparent at doses as low as 15 mg/kg/day. This easily excisable discrete tissue may be useful for investigating the effects of other compounds on adipose tissue reduction.

### Example 10

### Effect of NB-DNJ on Control Mouse Adipose Tissue

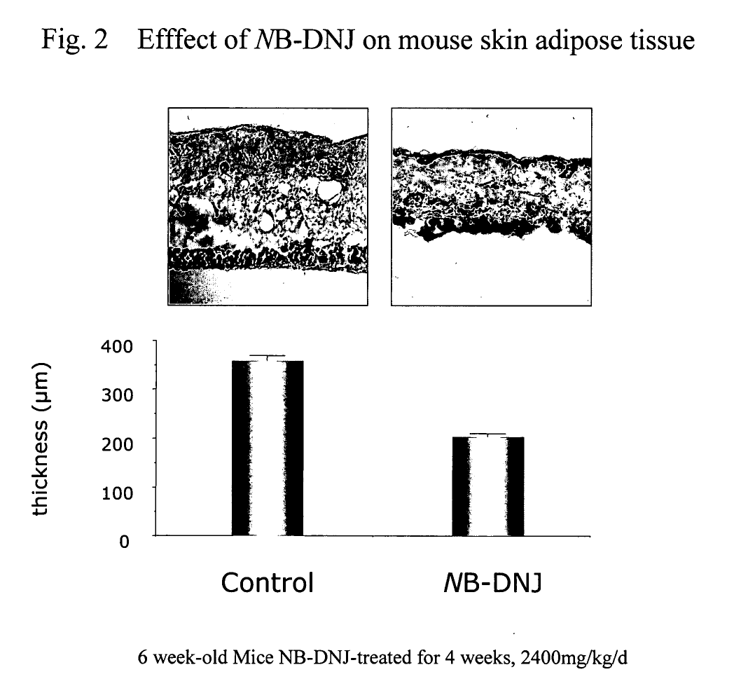

Gross dissection of the NB-DNJ-treated mice, revealed a significant depletion of white adipose tissue. Also, after complete depilation, this group of mice had transparent skin making the visceral organs, ventrally, and the kidneys and spinal vertebrae, dorsally, clearly visible in contrast to the opaque, non-transparent skin of controls. The reduction of subcutaneous white adipose tissue can be seen clearly in the histological sections of mouse skin (FIG. 2a.). The section from the treated mouse seems completely devoid of the fat visible in the subcutaneous layer in the control. This resulted in a 40% reduction in mouse skin thickness (FIG. 2b.).

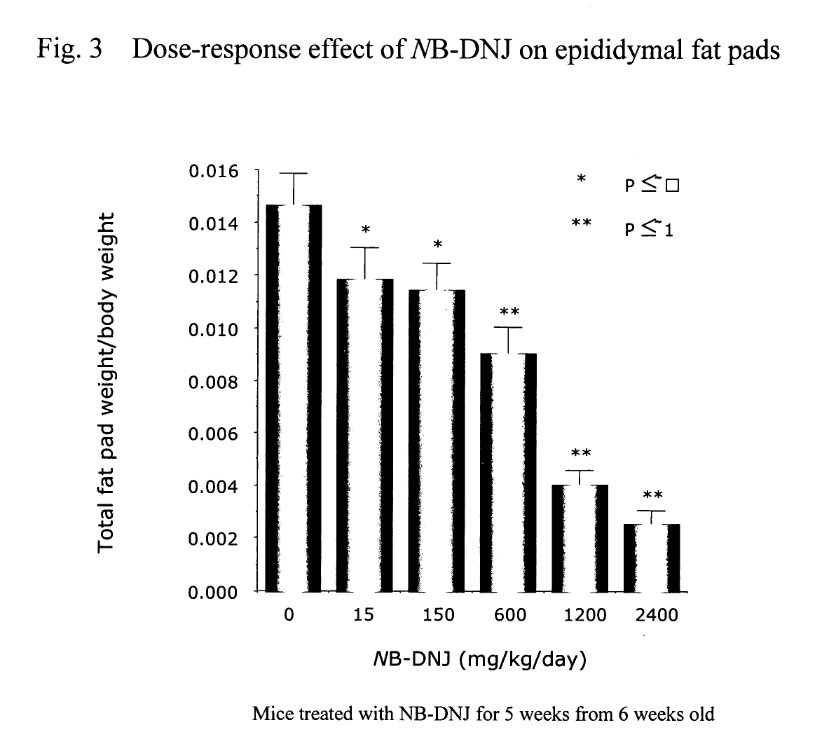

Further results on body fat depletion were obtained in the study on mouse epididymal fat pads (FIG. 3.). This discrete fat mass shows a clear dose-related response after five weeks treatment with NB-DNJ.

### Example 11

### Effect of NB-DNJ on Obese Mice

Leptin deficient ob/ob mice are widely used as an animal model of obesity and weigh more than twice as much as a normal mouse, even when fed the same diet. The mice have as much as a five-fold increase in fat content, are hyperphagic, hypothermic, have decreased energy expenditure and are unusually efficient at converting metabolic fuels into fat. Friedman & Halas, Nature 395, 763-70 (1998). These mice were used to investigate the effect of NB-DNJ. Results indicated that NB-DNJ induced appetite suppression, which greatly reduced growth rate in six week-old obese mice and caused weight loss in 12 week-old animals.

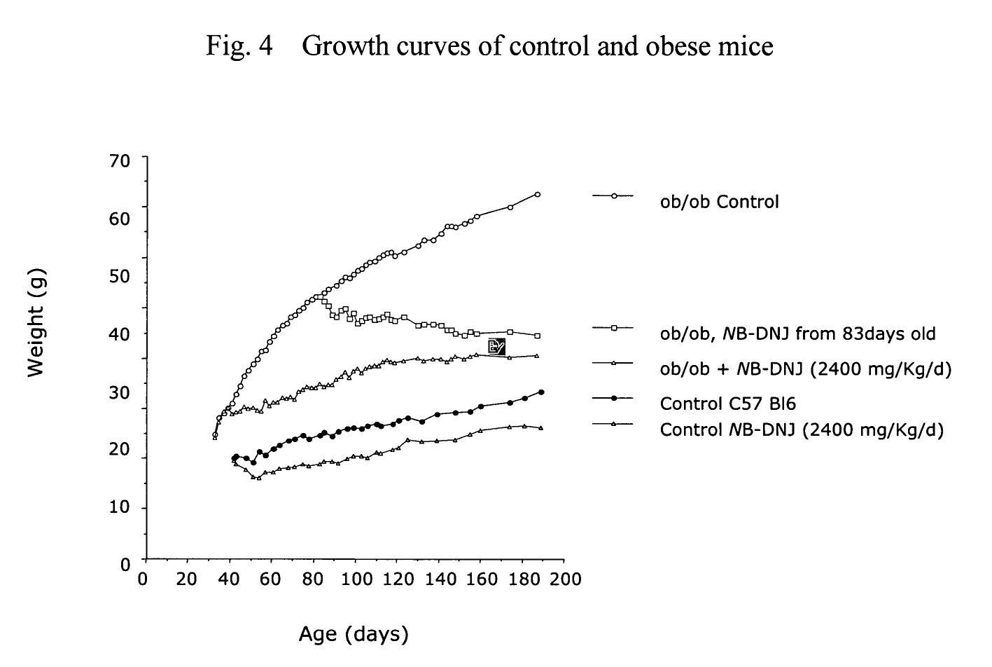

As NB-DNJ was so effective at reducing white adipose tissue in normal mice, a study was performed to discover if a similar effect would occur in obese mice. FIG. 4. shows the effect of long-term dosing on control C57B16 and ob/ob mice. The control obese mice grew from just over 20 g at five weeks old to about 60 g at six months of age whereas the lean controls had reached about 27 g at the same age. The group of obese mice started on NB-DNJ treatment at 7 weeks old grew at a very much reduced rate and weighed 35 g after six months (40% less than their control littermates). A further group of obese mice started on compound at 12 weeks old, lost weight over three months, stabilising their weight at a little less than 40 g.

### Example 12

### Effect of NB-DNJ on Lean Growth of Control Mice

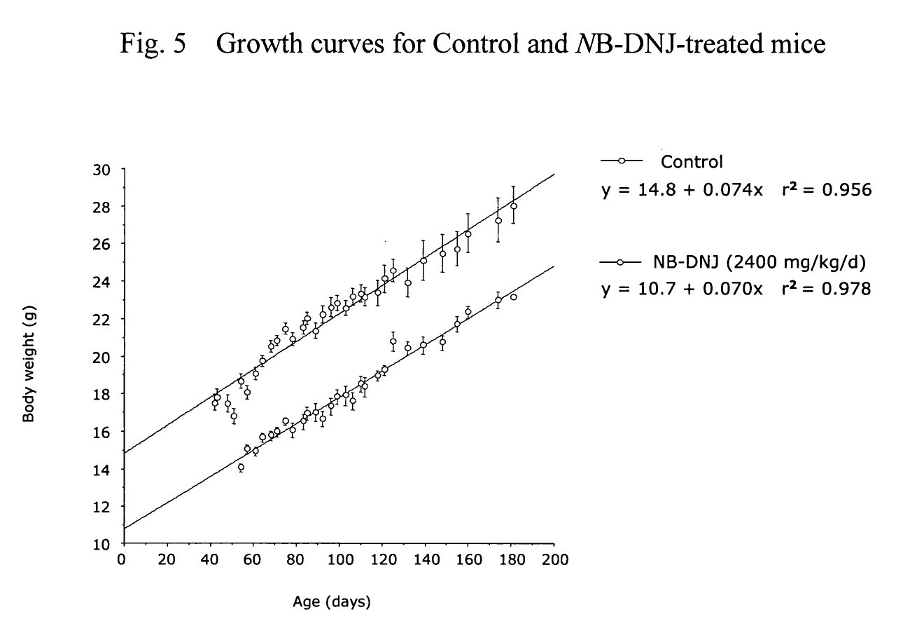

It can also be seen (FIG. 4) that the control mice lost weight over the first two weeks of treatment and then started to grow again, and continued to gain weight for the remainder of the study. FIG. 5. shows the growth of the control mice with and without NB-DNJ treatment excluding the portion of the curve where the treated mice lost weight. Growth in both groups of mice as measured by the gradient of the line is similar and the data were analysed by univariate analysis of variance (ANOVA) to compare the slopes of growth statistically. SPSS software was used to process the data and resulted in a p value of 0.487 for the difference between the slopes of the two growth curves. This result shows that, after the original weight loss, the mice then grow normally, albeit weighing 4 g less than their control counterparts. This indicates that the mice are losing most of their adipose tissue mass initially, and that the treatment has little or no effect on lean growth.

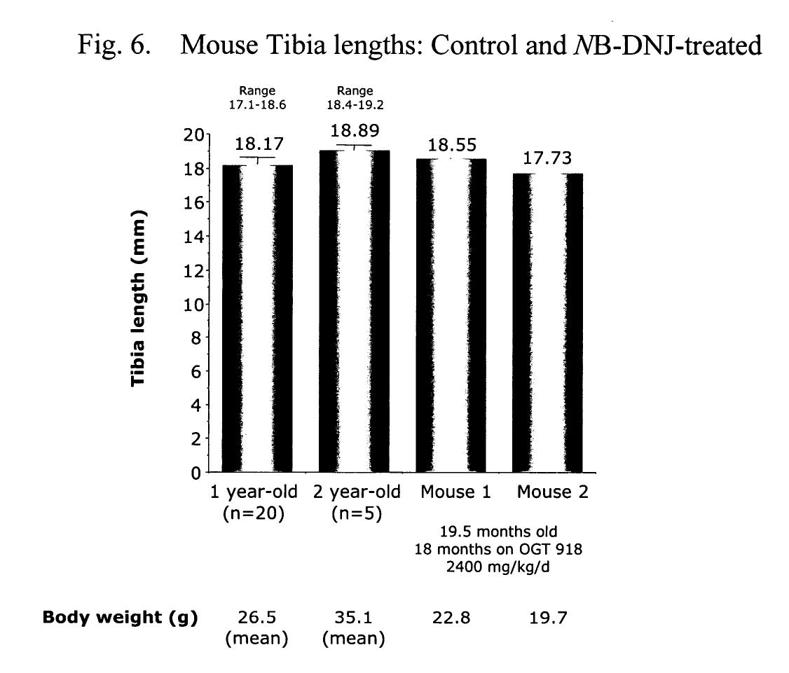

To establish further, whether NB-DNJ has an effect on lean growth of mice tibia lengths were measured in a large (n=20) group of control mice at one year of age, a smaller group (n=5) at two years of age and a comparison was made with two mice treated with NB-DNJ for 18 months. Tibia lengths in the treated mice were within the normal range (FIG. 6), which indicates that imino sugar-treatment had no effect on the lean growth of treated mice.

### Example 13

### Dietary Intake in Lean and Obese Mice

In an earlier study on the effects of NB-DNJ to deplete glycosphingolipids in mouse tissues, it was suggested that the reduction in body weight may be a result of appetite suppression. Platt et al., J Biol Chem 272, 19365-72. This hypothesis was tested by measuring dietary intake in both lean and obese mice fed on both powdered chow with or without imino sugar and with pelleted diet. To exclude any effects of the bitter taste of imino sugars and also as a means to bypass any disaccharidase inhibition in the gut, lean control and obese mice were given daily intraperitoneal injections of NB-DNJ for five and three weeks respectively. Also, a group of obese mice had NB-DNJ mini-pumps implanted and, after four weeks, when the pumps were empty, were then given intraperitoneal injections daily. This delivery route has the advantage that it allows easy assessment of dietary intake, as the mice can eat pelleted diet.

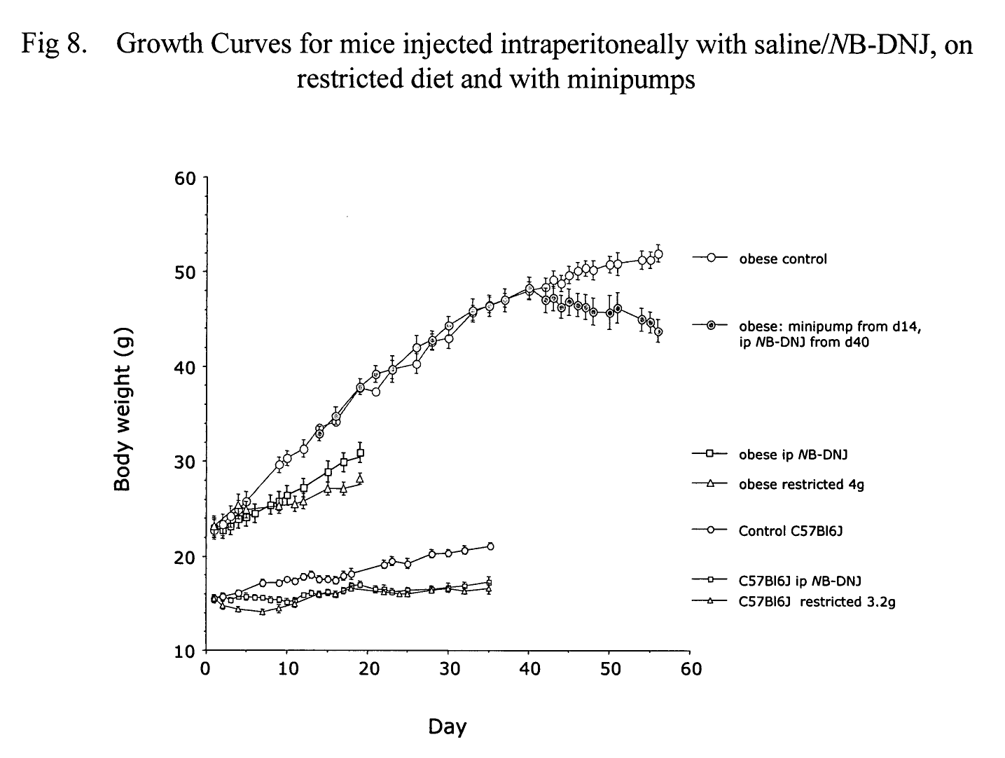

The results for dietary intake are presented in FIG. 7. In all the groups of mice, whether on a pelleted or powdered diet, NB-DNJ treatment caused a significant reduction in food intake of around 30%. Control lean mice ate almost 10% more pellet diet by weight than powdered diet whereas the obese mice ate 30% more pelleted diet on a daily basis. As a result of the larger intake of pelleted diet, the obese mice grew rather more rapidly than their powder-fed counterparts (results not shown). It is also notable that, though the obese mice fed on powdered chow ate the same quantity as control lean mice, the ob/ob phenotype resulted in the extra weight gain seen in FIG. 4. A further experiment was done to observe weight gain in control and obese mice fed a restricted diet comparable in quantity to that eaten by NB-DNJ treated. The mice were therefore provided with pellet diet weighing 30% less than normal ad libitum pellet intake. The reduced diet intake resulted in growth curves very similar to those for intraperitoneally-injected control and obese mice (FIG. 8). This implies that the caloric restriction as a result of appetite suppression is sufficient to cause the reduced growth/weight loss seen in drug-treated mice.

In summary, lean mice treated with N-butyldeoxynojirimycin (NB-DNJ), admixed with their diet, lost weight in the form of adipose tissue. Following the depletion of adipose tissue mass, the mice grew normally and did not have any reduction in lean mass. Obese mice treated with NB-DNJ also lost weight or gained weight at a greatly reduced rate compared to non-treated controls. Both the lean and obese groups of mice treated with NB-DNJ ate up to one third less than untreated controls. Mice treated with the N-substituted galactose imino sugar analogue (NB-DGJ) did not lose weight.

Additional embodiments are within the scope of the invention. For example, the invention is further illustrated by the following numbered embodiments:
1. A composition for treating obesity comprising an N-substituted imino sugar and a pharmaceutically acceptable excipient.
2. The composition of embodiment 1, wherein said N-substituted imino sugar is N-butyldeoxynojirimycin.
3. A method for treating obesity comprising administering an effective amount of an N-substituted imino sugar that does not cause an initial hyperphagic response.
4. The method of embodiment 3, wherein said N-substituted imino sugar is of N-butyldeoxynojirimycin.
5. A method for centrally suppressing an appetite comprising administering an effective amount of N-butyldeoxynojirimycin to a subject.
6. The method of embodiment 5, wherein said subject is obese.
7. The method of embodiment 5 or 6, wherein said N-butyldeoxynojirimycin caused a reduction in food intake sufficient to cause weight loss.
8. A method for depleting white adipose tissue in a subject comprising administering an effective amount of N-butyldeoxynojirimycin.
9. The method of embodiment 8, wherein said subject is obese.

1. A composition for treating obesity comprising an N-substituted imino sugar and a pharmaceutically acceptable excipient.

2. The composition of claim 1, wherein said N-substituted imino sugar is N-butyldeoxynojirimycin.

3. A method for treating obesity comprising administering an effective amount of an N-substituted imino sugar that does not cause an initial hyperphagic response.

4. The method of claim 3, wherein said N-substituted imino sugar is of N-butyldeoxynojirimycin.

5. A method for centrally suppressing an appetite comprising administering an effective amount of N-butyldeoxynojirimycin to a subject.

6. The method of claim 5, wherein said subject is obese.

7. The method of claim 5, wherein said N-butyldeoxynojirimycin caused a reduction in food intake sufficient to cause weight loss.

8. The method of claim 6, wherein said N-butyldeoxynojirimycin caused a reduction in food intake sufficient to cause weight loss.

9. A method for depleting white adipose tissue in a subject comprising administering an effective amount of N-butyldeoxynojirimycin.

10. The method of claim 9, wherein said subject is obese.
